(19) United States
(12) Patent Application Publication
(10) Pub. No.: US 2021/0030595 A1
(43) Pub. Date: Feb. 4, 2021

(54) MEDICAL DEVICE FOR SUPPORT AND FIXATION

(71) Applicant: YUEH-MING LIU, Taichung City (TW)

(72) Inventor: YUEH-MING LIU, Taichung City (TW)

(21) Appl. No.: 16/524,260

(22) Filed: Jul. 29, 2019

(51) Int. Cl. A61F 13/00; A61F 5/01; A61F 13/10

(52) U.S. Cl. CPC A61F 13/00046; A61F 5/0118; A61F 13/00017; A61F 13/108; A61F 13/00012; A61F 13/00029

(57) ABSTRACT

The medical device for support and fixation in accordance has at least one supporting layer, at least two three-dimensional fiber webs, and a coating material. The at least one supporting layer has multiple air vents disposed thereon. The at least two three-dimensional fiber webs are mounted to two sides of the at least one supporting layer, which makes the medical device for support and fixation a flexible structure. Each one of the at least two three-dimensional fiber webs has multiple fiber web units disposed adjacently. Each one of the multiple fiber web units has a major through hole and multiple fibers. The major through hole is formed through the fiber web unit. The multiple fibers are braided to form the fiber web unit and surround the major through hole. The coating material is coated on the at least two three-dimensional fiber webs.

### BACKGROUND OF THE INVENTION

### 1. Field of the Invention

The present invention relates to a medical device, and more particularly to a medical device for support and fixation.

### 2. Description of Related Art

Conventional medical devices for support and fixation, such as a splint for hand fracture or leg fracture and a scoliosis brace, are mainly made of material such as plastic and plaster. Since plastic and plaster are both airproof material, bed sores may develop on the skin that is covered by the conventional medical devices for a long time. Furthermore, conventional medical devices made of plastic and plaster are heavy, which restricts the patients' mobility. Moreover, the making of the conventional medical devices often involves mold developing or 3D printing, which costs much and takes much time in production.

To overcome the shortcomings of the conventional medical supplies, the present invention tends to provide a medical device for support and fixation to mitigate or obviate the aforementioned problems.

### SUMMARY OF THE INVENTION

The main objective of the present invention is to provide a medical device for support and fixation.

The medical device for support and fixation in accordance with the present invention has at least one supporting layer, at least two three-dimensional fiber webs, and a coating material. The at least one supporting layer has multiple air vents disposed at spaced intervals, and each one of the multiple air vents is a polygon. The at least two three-dimensional fiber webs are mounted to two sides of the at least one supporting layer respectively, which makes the medical device for support and fixation a flexible structure. Each one of the at least two three-dimensional fiber webs has multiple fiber web units disposed adjacently. Each one of the multiple fiber web units has a major through hole and multiple fibers. The major through hole is formed through the fiber web unit and communicates with at least one of the multiple air vents of the at least one supporting layer. The multiple fibers are braided to form the fiber web unit and surround the major through hole of the fiber web unit. Each one of the multiple fibers is braided by multiple fiber threads and has multiple minor through holes. Each one of the multiple minor through holes communicates with at least one of the multiple air vents of the at least one supporting layer. The coating material is coated on the at least two three-dimensional fiber webs, and infiltrates to the at least one supporting layer, which makes the medical device for support and fixation waterproof.

Other objectives, advantages and novel features of the invention will become more apparent from the following detailed description when taken in conjunction with the accompanying drawings.

### BRIEF DESCRIPTION OF THE DRAWINGS

FIG. 1 is an exploded perspective view of a first embodiment of a medical device for support and fixation in accordance with the present invention;

FIG. 2 is a top view in partial section of the medical device for support and fixation in FIG. 1;

FIG. 3 is an enlarged cross-sectional side view of the medical device for support and fixation in FIG. 1;

FIG. 4 is an operational top view of the medical device for support and fixation in FIG. 1;

FIG. 5 is another operational top view of the medical device for support and fixation in FIG. 1;

FIG. 6 is an exploded perspective view of a second embodiment of a medical device for support and fixation in accordance with the present invention;

FIG. 7A is an operational top view of the medical device for support and fixation in FIG. 6;

FIG. 7B is an enlarged top view of the medical device for support and fixation in FIG. 7A;

FIG. 8A is a cross-sectional side view of the medical device for support and fixation in FIG. 6;

FIG. 8B is an enlarged side view of the medical device for support and fixation in FIG. 8A;

FIG. 9 is an exploded perspective view of a third embodiment of a medical device for support and fixation in accordance with the present invention;

FIG. 10 is an operational front view of the medical device for support and fixation in FIG. 9; and

FIG. 11 is an operational back view of the medical device for support and fixation in FIG. 9.

### DETAILED DESCRIPTION OF THE PREFERRED EMBODIMENT

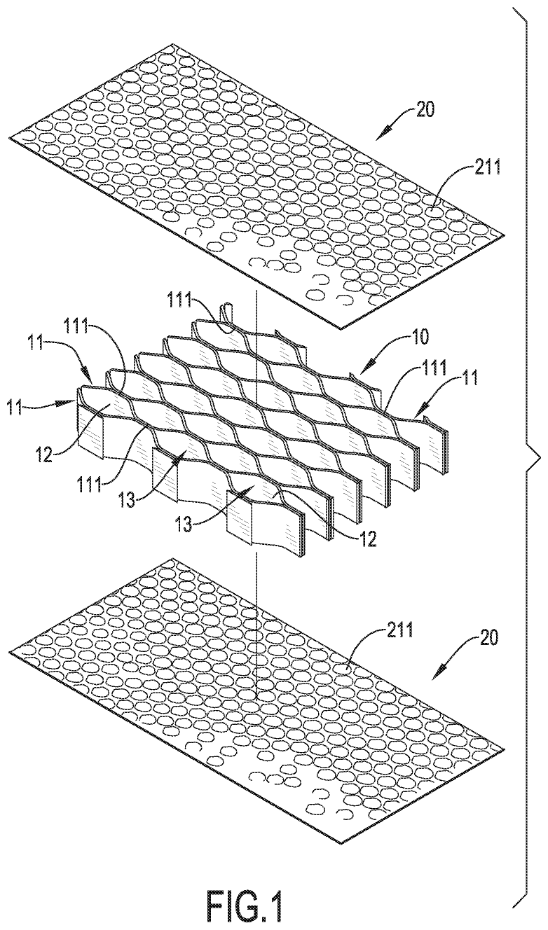

With reference to FIGS. 1 to 3, a first embodiment of a medical device for support and fixation in accordance with the present invention comprises at least one supporting layer 10, at least two three-dimensional fiber webs 20, and a coating material 30.

With reference to FIG. 1, the at least one supporting layer 10 has multiple supporting elements 11, multiple air vents 12, and multiple supporting units 13. The multiple supporting elements 11 are disposed adjacently, and each one of the multiple supporting elements 11 has a thickness, a longitudinal direction, two side surfaces, and multiple connecting portions 111. The multiple supporting elements 11 extend along a same direction. The two side surfaces of the supporting element 11 are disposed on two sides of the supporting elements 11 and face two opposite directions respectively. Each one of the multiple connecting portions 111 is disposed on one of the two side surfaces of the supporting element 11, and the multiple connecting portions 111 are disposed at spaced intervals. Each one of the multiple connecting portions 111 is disposed on one of the two side surfaces of the supporting element 11 opposite to the side on which an adjacent connecting portion 111 is disposed. Each one of the multiple connecting portions 111 on the supporting element 11 is connected to one of the multiple connecting portions 111 on an adjacent supporting element 11, and the two corresponding connecting portions 111 form a connecting point.

Since each one of the multiple connecting portions 111 of the supporting element 11 is disposed at a spaced interval with the adjacent one of the multiple connecting portions 111 on the same side surface of the supporting element 11, an air vent 12 is formed between two adjacent connecting points of two adjacent supporting elements 11. The multiple air vents 12 are disposed at spaced intervals on the at least one supporting layer 10, which makes the at least one supporting layer 10 lightweight and structurally dense. Two of the multiple supporting elements 11 around each one of the multiple air vents 12 form one of the multiple supporting units 13. The multiple supporting units 13 are disposed at spaced intervals on the at least one supporting layer 10, and each one of the multiple air vents 12 is located in one of the multiple supporting units 13.

In the present invention, each one of the multiple supporting units 13 is a polygon. Moreover, each one of the multiple supporting units 13 is a hexagon, such that the multiple supporting units 13 are arranged as a honeycomb. Because each one of the multiple supporting units 13 is hexagonal, forces born by each side of each one of the supporting units 13 are the same. Therefore, each one of the multiple supporting units 13 is capable of distributing pressure. Having the supporting units 13, the at least one supporting layer 10 is capable of sustaining stress and cushioning. Overall, the at least one supporting layer 10 is breathable, lightweight and structurally dense, and being capable of sustaining stress and cushioning. Besides, the at least one supporting layer 10 is a paper honeycomb.

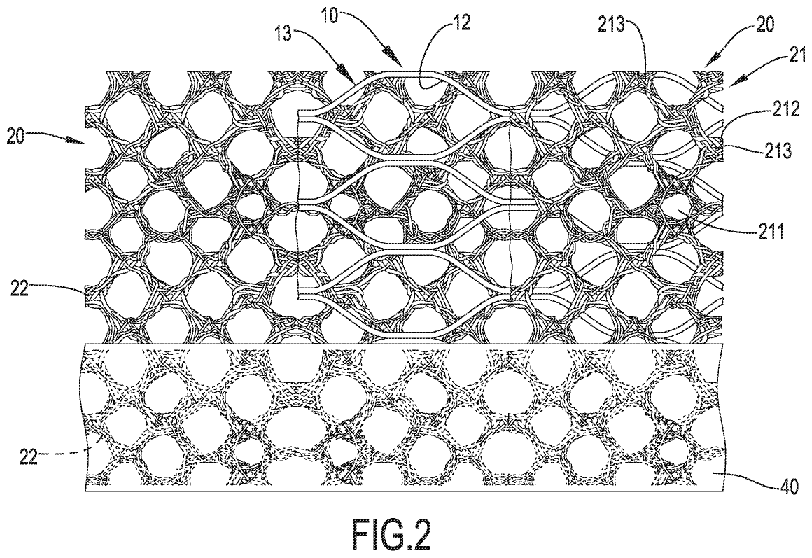

With reference to FIGS. 1 and 2, the at least two three-dimensional fiber webs 20 are mounted to the two sides of the at least one supporting layer 10 respectively, which makes the medical device for support and fixation a flexible structure. Since each one of the supporting elements 11 of the at least one supporting layer 10 has the thickness, the at least two three-dimensional fiber webs 20 are disposed at a spaced interval. Each one of the at least two three-dimensional fiber webs 20 has multiple fiber web units 21, and the multiple fiber web units 21 are disposed adjacently on the three-dimensional fiber web 20. Each one of the multiple fiber web units 21 has a major through hole 211 and multiple fibers 212. The major through hole 211 is formed through the fiber web unit 21 and communicates with at least one of the multiple air vents 12 of the at least one supporting layer 10. The multiple fibers 212 are braided to form the fiber web unit 21 and surround the major through hole 211 of the fiber web unit 21. Each one of the multiple fibers 212 is braided by multiple fiber threads and has multiple minor through holes 213. Each one of the multiple minor through holes 213 communicates with at least one of the multiple air vents 12 of the at least one supporting layer 10. Sizes of the minor through holes 213 can be different from each other. The size of each one of the minor through holes 213 of the fiber 212 is smaller than a size of each one of the major through holes 211 of the three-dimensional fiber web 20. In the present invention, a contour of each one of the major through holes 211 of the multiple fiber web units 21 is substantially hexagonal. Moreover, each one of the multiple fiber threads is made of polyester.

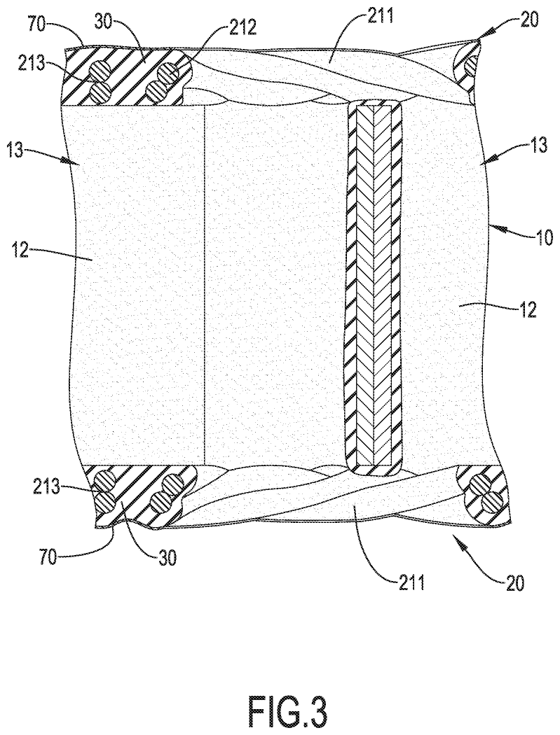

With reference to FIG. 3, the coating material 30 is coated on the at least two three-dimensional fiber webs 20, and infiltrates to an inner surface of each one of the multiple supporting units 13 of the at least one supporting layer 10. The coating material 30 is composed of 18% to 28% water, 0.1% to 1% hydrogenated light paraffin, 1% to 10% amorphous silicon dioxide, 2% to 12% 2-hydroxyethyl acrylate, styrene, acrylic acid, and poly(methyl methacrylate) (PMMA), and 43% to 53% acrylic copolymer by weight. The coating material 30 is moisture-resistant, wear resistant, scratch resistant, uv-resistant (ultraviolent radiation), anticorrosive, cracking-preventive, and good in adherence. The coating material 30 can penetrate through the multiple major through holes 211 and the multiple minor through holes 213 of each one of the at least two three-dimensional fiber webs 20 and infiltrates to the inner surface of each one of the multiple supporting units 13 of the at least one supporting layer 10. Each one of the multiple fibers 212 is braided by the multiple fiber threads, and each one of the multiple fiber threads is made of polyester. Being made of polyester, each one of the multiple fiber threads can easily absorb the coating material 30. The coating material 30 in turn infiltrates to the inner surface of each one of the multiple supporting units 13 of the at least one supporting layer 10. The coating material 30 can be coated on the at least two three-dimensional fiber webs 20 and the at least one supporting layer 10, and thereby the medical device for support and fixation is waterproof.

Besides, since each one of the multiple fiber threads is made of polyester, each one of the at least two three-dimensional fiber webs 20 has a soft texture. Even though the multiple air vents 12 make the at least one supporting layer 10 uneven at its side surfaces, the at least two three-dimensional fiber webs 20 are still mounted firmly to the two sides of the at least one supporting layer 10. Furthermore, the medical device for support and fixation comprises a curing agent. The curing agent is coated on the at least two three-dimensional fiber webs 20 of the medical device for support and fixation in order to enhance the structural strength of each one of the at least two three-dimensional fiber webs 20.

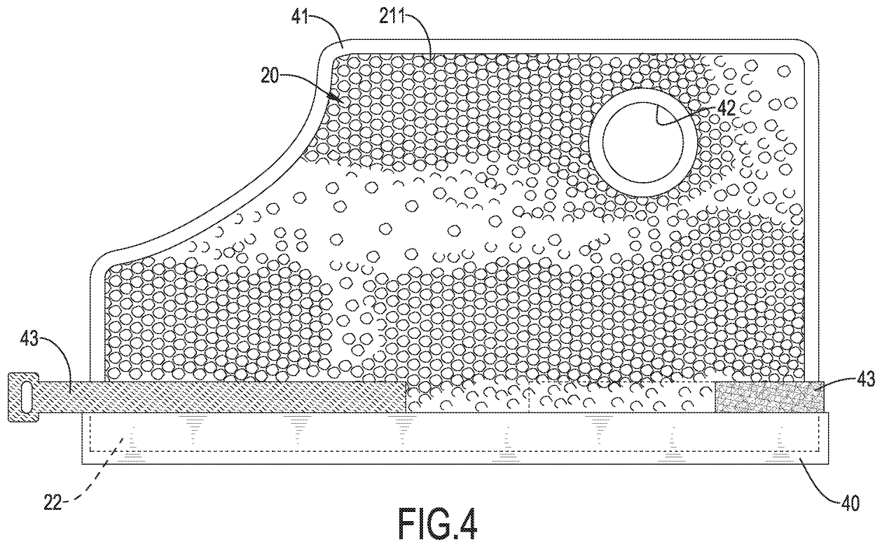

Moreover, with reference to FIGS. 2 and 4, each one of the at least two three-dimensional fiber webs 20 has four edges, and one of the four edges extends out of the at least one supporting layer 10 and forms an extending portion 22. Since the at least two three-dimensional fiber webs 20 are mounted to the two sides of the at least one supporting layer 10 respectively, the two extending portions 22 of the two three-dimensional fiber webs 20 overlap each other. A label layer 40 can be wrapped or riveted on the two overlapping extending portions 22 of the two three-dimensional fiber webs 20, and the label layer 40 may be an advertising label. Besides, the four edges of each one of the at least two three-dimensional fiber webs 20 all extend out of the at least one supporting layer 10 and form four extending portions 22. Users can wrap a decorating layer 41 on three groups of the overlapping extending portions 22 of the two three-dimensional fiber webs 20 except a group of the overlapping extending portions 22 of the two three-dimensional fiber webs 20 wrapped with the label layer 40.

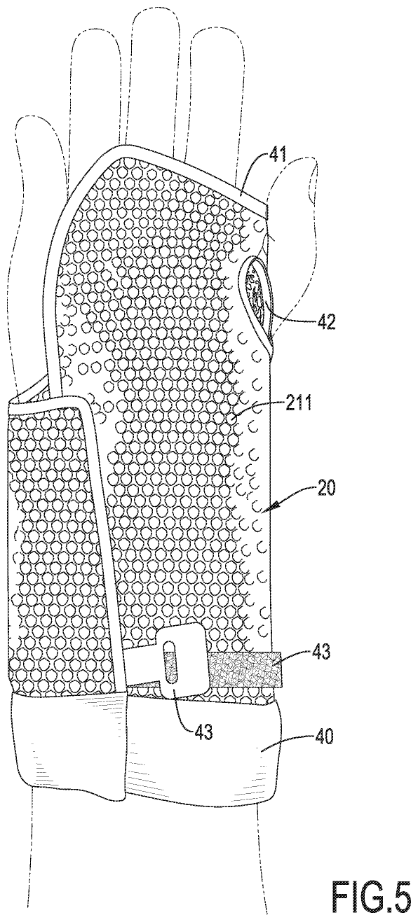

The first embodiment of the medical device for support and fixation in accordance with the present invention can be wrapped around limbs of a patient. When the medical device for support and fixation is served as a splint for a hand and a wrist, a passing hole 42 is disposed on the medical device for support and fixation. The passing hole 42 is formed through each one of the at least two three-dimensional fiber webs 20 and the at least one supporting layer 10. In use, place a thumb of a patient into the passing hole 42 as shown in FIG. 5, and then wrap other parts of the medical device for support and fixation around a hand and a wrist of the patient. In addition, the medical device for support and fixation can be utilized with multiple fixing elements 43. Each one of the multiple fixing elements 43 is mounted to one of the at least two three-dimensional fiber webs 20. By winding the medical device for support and fixation and connecting one of the multiple fixing elements 43 with a corresponding one of the multiple fixing elements 43, the medical device for support and fixation can remain wrapped around the hand and wrist of the patient. Each one of the multiple fixing elements 43 may be a button, an elastic belt, or a hook-and-loop fastener.

By mounting the at least two three-dimensional fiber webs 20 to the two sides of the at least one supporting layer 10, and coating the coating material 30 on the at least two three-dimensional fiber webs 20, the medical device for support and fixation is waterproof since the coating material 30 infiltrates to the inner surface of each one of the multiple supporting units 13 of the at least one supporting layer 10. As the at least one supporting layer 10 has multiple air vents 12 and each one of the at least two three-dimensional fiber webs 20 has multiple major through holes 211 and multiple minor through holes 213, the medical device for support and fixation is breathable. In addition, the at least one supporting layer 10 and the at least two three-dimensional fiber webs 20 are lightweight, which makes the medical device for support and fixation lightweight as well. Though made up of materials low in cost, the medical device for support and fixation has the ability to sustain stress and to cushion patients against impact because the at least one supporting layer 10 is capable of sustaining stress and cushioning. At the same time, the medical device for support and fixation can overcome the disadvantage that the conventional medical device for support and fixation is unbreathable and heavy.

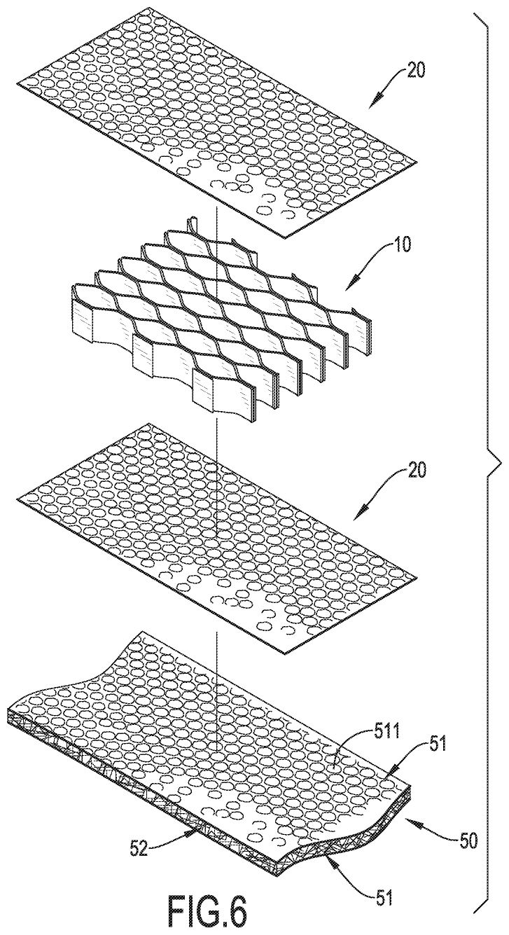

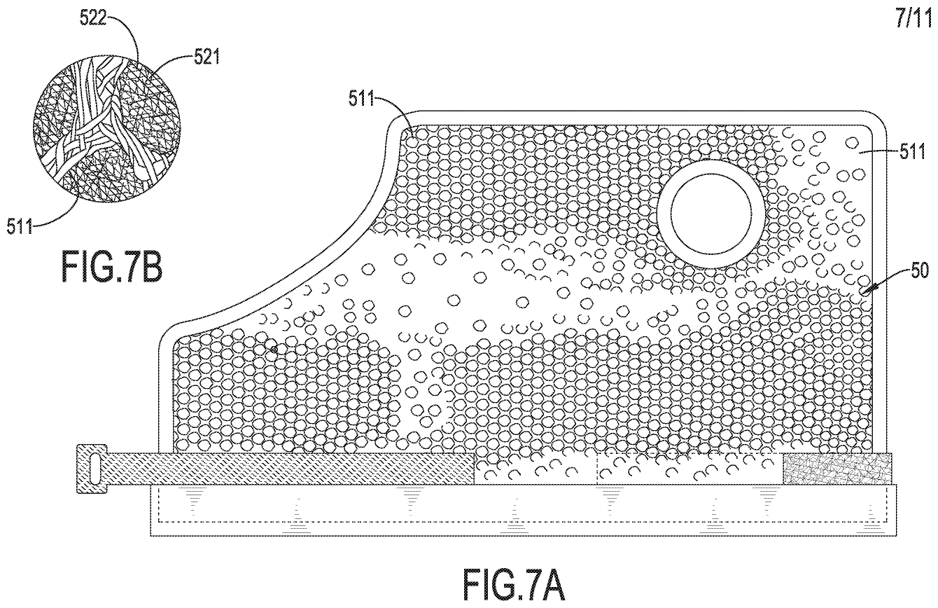

With reference to FIGS. 6, 7A, 7B, 8A, and 8B, a second embodiment of a medical device for support and fixation in accordance with the present invention is substantially the same as the first embodiment, and the difference between the second embodiment and the first embodiment is that the second embodiment comprises a multi-layered medical elastic breathable fabric 50. The multi-layered medical elastic breathable fabric 50 is mounted to one of the at least two three-dimensional fiber webs 20 and has two surface layers 51 and a cushioning layer 52. The two surface layers 51 are disposed at a spaced interval, and each one of the two surface layers 51 is made of polyethylene (PE). Polyethylene is odorless, non-toxic, and has a low permeable rate. Each one of the two surface layers 51 has multiple vents 511 disposed at spaced intervals. Each one of the multiple vents 511 is formed through the surface layer 51. The cushioning layer 52 is disposed between the two surface layers 51 and has multiple cushioning layer fiber threads 521 and multiple openings 522. Each one of the multiple cushioning layer fiber threads 521 is made of polyethylene. The multiple cushioning layer fiber threads 521 are wound to form the cushioning layer 52 and the multiple openings 522. The multiple openings 522 are disposed separately among the multiple cushioning layer fiber threads 521. Air can be contained in the multiple openings 522, and the cushioning layer 52 in turn obtain cushioning effect and softness. In addition, each one of the two surface layers 51 and each one of the cushioning layer fiber threads 521 may be made of polyester.

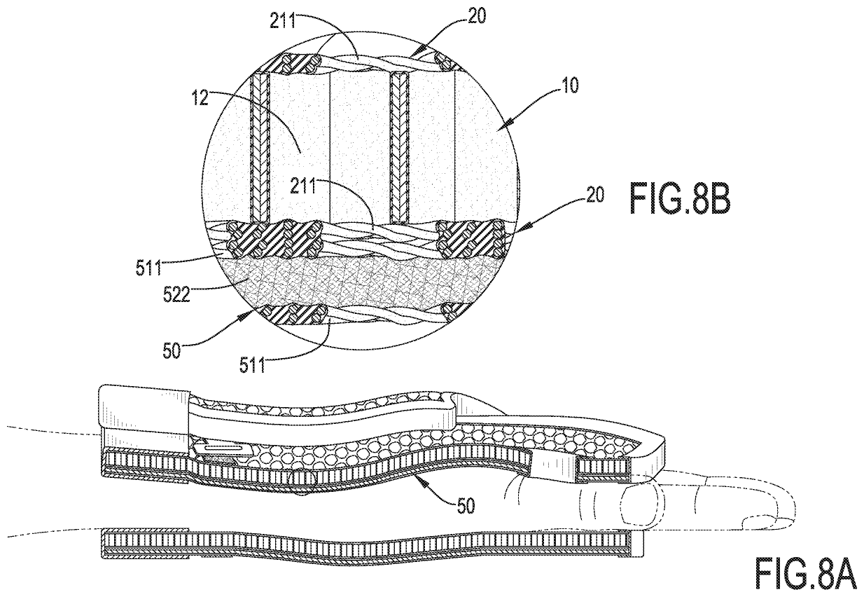

In use, the multi-layered medical elastic breathable fabric 50 is oriented toward a hand or a leg of a human body as shown in FIG. 8A and 8B, and the second embodiment of the medical device for support and fixation is wrapped around the hand or the leg of the human body. Patients can wear the medical device for support and fixation comfortably because the multi-layered medical elastic breathable fabric 50 has the cushioning effect and softness and the at least two three-dimensional fiber webs 20 does not contact the skin of the patients directly. Since the multi-layered medical elastic breathable fabric 50 has multiple vents 511 and multiple openings 522, the medical device for support and fixation is still breathable though the multi-layered medical elastic breathable fabric 50 is added. In addition, because polyethylene is odorless, non-toxic, and has a low permeable rate, the multi-layered medical elastic breathable fabric 50 is harmless to the human body and waterproof.

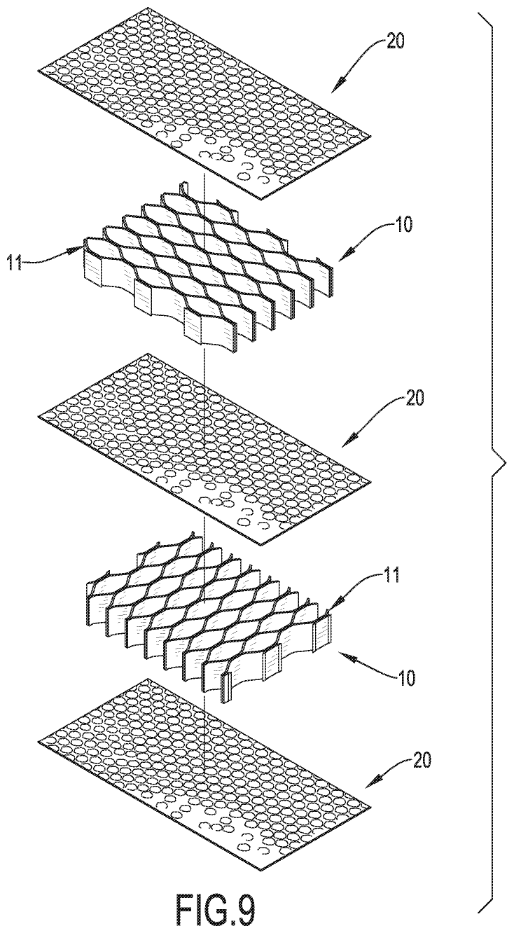

With reference to FIG. 9, a third embodiment of a medical device for support and fixation in accordance with the present invention comprises two supporting layers 10 and three three-dimensional fiber webs 20. The three three-dimensional fiber webs 20 are disposed at spaced intervals. Each one of the two supporting layers 10 is disposed between two adjacent ones of the three three-dimensional fiber webs 20 of the three three-dimensional fiber webs 20, and thereby the two supporting layers 10 and three three-dimensional fiber webs 20 are arranged in an alternate order. The longitudinal direction of each one of the supporting elements 11 of one of the two supporting layers 10 intersects with the longitudinal direction of each one of the supporting elements 11 of the other one of the two supporting layers 10 and define an included angle. Furthermore, the included angle between the two corresponding supporting elements 11 is 90°. The structural strength of the medical device for support and fixation is therefore enhanced.

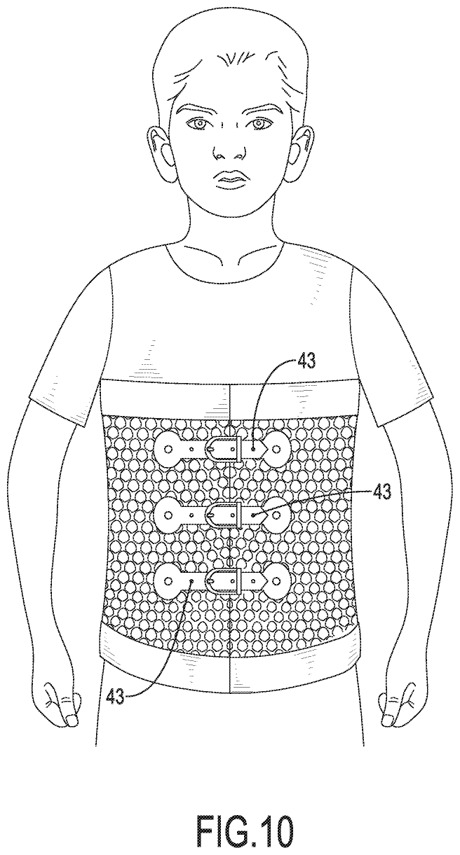

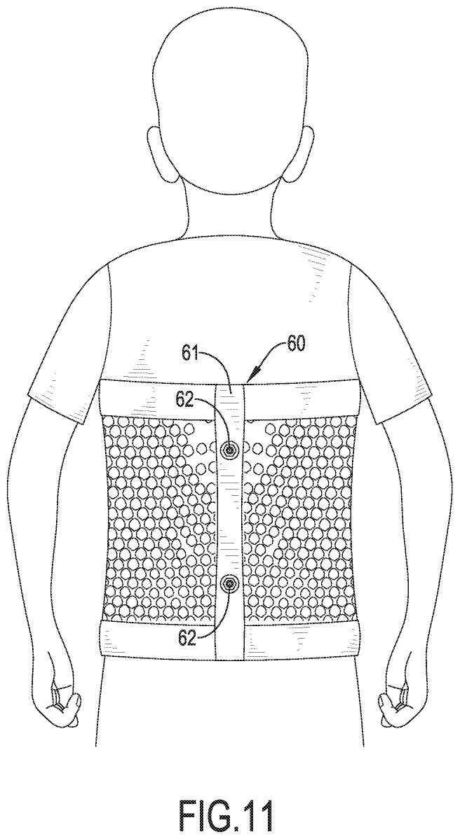

With reference to FIGS. 10 and 11, in use, the third embodiment of the present invention can be wrapped around a torso of a patient. The third embodiment can be utilized with multiple fixing elements 43, too. Furthermore, the third embodiment of the present invention can be utilized with a supporting brace 60. The supporting brace 60 is combined with the medical device for support and fixation, and has a bracing element 61 and a fastener 62. The bracing element 61 is vertically disposed on the supporting brace 60, and a position of the bracing element 61 corresponds to that of a spine of a human body. The fastener 62 is mounted through the medical device for support and fixation and the bracing element 61, and fixes the bracing element 61 to the supporting brace 60. By combining the supporting brace 60 with the medical device for support and fixation, the present invention can be utilized in treatment of scoliosis.

With the aforementioned features and technical characteristics of the present invention, the medical device for support and fixation has the following advantages.

1. The multiple air vents 12 of the at least one supporting layer 10, the multiple major through holes 211 and the multiple minor through holes 213 of each one of the at least two three-dimensional fiber webs 20, and the multiple vents 511 and the multiple openings 522 of the multi-layered medical elastic breathable fabric 50 make the medical device for support and fixation a breathable structure though the present invention has multiple layers. The drawback that the conventional medical devices for support and fixation are airproof and sores may develop on skin is solved thereby.

2. Since each one of the supporting layers 10, each one of the three-dimensional fiber webs 20, and the multi-layered medical elastic breathable fabric 50 are lightweight and structurally dense, the present invention is therefore lightweight and structurally dense.

3. Each one of the supporting layers 10, each one of the three-dimensional fiber webs 20, and the multi-layered medical elastic breathable fabric 50 are materials low in cost, which effectively reduces the cost for making the medical device for support and fixation. Also, since the present invention is easy to produce, time taken to produce the present invention is less than time taken to produce the conventional medical devices for support and fixation involving mold developing or 3D printing.

4. The coating material 30 is coated on the at least two three-dimensional fiber webs 20, and infiltrates to the inner surface of each one of the multiple supporting units 13 of the at least one supporting layer 10. The coating material 30 is moisture-resistant, so the present invention is waterproof. Furthermore, the multi-layered medical elastic breathable fabric 50 is made of polyethylene, which also makes the present invention waterproof.

5. By disposing the at least one supporting layer 10 capable of sustaining stress and cushioning, the present invention is capable of sustaining stress and cushioning, too.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A medical device for support and fixation comprising:
at least one supporting layer having
multiple air vents disposed at spaced intervals, and each one of the multiple air vents being a polygon; and
two sides;
at least two three-dimensional fiber webs mounted to the two sides of the at least one supporting layer respectively and making the medical device for support and fixation a flexible structure, and each one of the at least two three-dimensional fiber webs having
multiple fiber web units disposed adjacently, and each one of the multiple fiber web units having
a major through hole formed through the fiber web unit and communicating with at least one of the multiple air vents of the at least one supporting layer; and
multiple fibers braided to form the fiber web unit, surrounding the major through hole, each one of the multiple fibers being braided by multiple fiber threads having
multiple minor through holes, and each one of the multiple minor through holes communicating with at least one of the multiple air vents of the at least one supporting layer; and
a coating material coated on the at least two three-dimensional fiber webs, and infiltrating to the at least one supporting layer and making the medical device for support and fixation waterproof.

2. The medical device for support and fixation as claimed in claim 1, wherein the medical device for support and fixation has
two said supporting layers; and
three said three-dimensional fiber webs arranged in an alternate order with the two supporting layers, wherein the two supporting layers are at outer sides of the medical device for support and fixation.

3. The medical device for support and fixation as claimed in claim 2, wherein the coating material is composed of 18% to 28% water, 0.1% to 1% hydrogenated light paraffin, 1% to 10% amorphous silicon dioxide, 2% to 12% 2-hydroxyethyl acrylate, styrene, acrylic acid, and poly(methyl methacrylate) (PMMA), and 43% to 53% acrylic copolymer by weight.

4. The medical device for support and fixation as claimed in claim 3, wherein each one of the supporting layers has multiple supporting elements, and each one of the multiple supporting elements has a longitudinal direction intersecting with the longitudinal direction of each one of the supporting elements of the other one of the two supporting layers and define an included angle.

5. The medical device for support and fixation as claimed in claim 1, wherein the medical device for support and fixation comprises a multi-layered medical elastic breathable fabric mounted to one of the at least two three-dimensional fiber webs and having
two surface layers disposed at a spaced interval, each being made of polyethylene, and each having
multiple vents disposed at spaced intervals and each formed through the surface layer; and
a cushioning layer disposed between the two surface layers and having
multiple cushioning layer fiber threads wound to form the cushioning layer, and each made of polyethylene; and
multiple openings disposed separately among the multiple cushioning layer fiber threads.

6. The medical device for support and fixation as claimed in claim 1, wherein the medical device for support and fixation comprises a multi-layered medical elastic breathable fabric mounted to one of the at least two three-dimensional fiber webs and having
two surface layers disposed at a spaced interval, each being made of polyester, and each having
multiple vents disposed at spaced intervals and each formed through the surface layer; and
a cushioning layer disposed between the two surface layers and having
multiple cushioning layer fiber threads wound to form the cushioning layer, and each made of polyester; and
multiple openings disposed separately among the multiple cushioning layer fiber threads.

7. The medical device for support and fixation as claimed in claim 2, wherein the medical device for support and fixation comprises a multi-layered medical elastic breathable fabric mounted to one of the two three-dimensional fiber webs and having
two surface layers disposed at a spaced interval, each being made of polyethylene, and each having
multiple vents disposed at spaced intervals and each formed through the surface layer; and
a cushioning layer disposed between the two surface layers and having
multiple cushioning layer fiber threads wound to form the cushioning layer, and each made of polyethylene; and
multiple openings disposed separately among the multiple cushioning layer fiber threads.

8. The medical device for support and fixation as claimed in claim 2, wherein the medical device for support and fixation comprises a multi-layered medical elastic breathable fabric mounted to one of the two three-dimensional fiber webs and having
two surface layers disposed at a spaced interval, each being made of polyester, and each having
multiple vents disposed at spaced intervals and each formed through the surface layer; and
a cushioning layer disposed between the two surface layers and having multiple cushioning layer fiber threads wound to form the cushioning layer, and each made of polyester; and
multiple openings disposed separately among the multiple cushioning layer fiber threads.

9. The medical device for support and fixation as claimed in claim 3, wherein the medical device for support and fixation comprises a multi-layered medical elastic breathable fabric mounted to one of the two three-dimensional fiber webs and having
two surface layers disposed at a spaced interval, each being made of polyethylene, and each having
multiple vents disposed at spaced intervals and each formed through the surface layer; and
a cushioning layer disposed between the two surface layers and having
multiple cushioning layer fiber threads wound to form the cushioning layer, and each made of polyethylene; and
multiple openings disposed separately among the multiple cushioning layer fiber threads.

10. The medical device for support and fixation as claimed in claim 2, wherein the medical device for support and fixation comprises a multi-layered medical elastic breathable fabric mounted to one of the two three-dimensional fiber webs and having
two surface layers disposed at a spaced interval, each being made of polyester, and each having
multiple vents disposed at spaced intervals and each formed through the surface layer; and
a cushioning layer disposed between the two surface layers and having
multiple cushioning layer fiber threads wound to form the cushioning layer, and each made of polyester; and
multiple openings disposed separately among the multiple cushioning layer fiber threads.

11. The medical device for support and fixation as claimed in claim 3, wherein the medical device for support and fixation comprises a multi-layered medical elastic breathable fabric mounted to one of the two three-dimensional fiber webs and having
two surface layers disposed at a spaced interval, each being made of polyethylene, and each having
multiple vents disposed at spaced intervals and each formed through the surface layer; and
a cushioning layer disposed between the two surface layers and having
multiple cushioning layer fiber threads wound to form the cushioning layer, and each made of polyethylene; and
multiple openings disposed separately among the multiple cushioning layer fiber threads.

12. The medical device for support and fixation as claimed in claim 2, wherein the medical device for support and fixation comprises a multi-layered medical elastic breathable fabric mounted to one of the two three-dimensional fiber webs and having
two surface layers disposed at a spaced interval, each being made of polyester, and each having
multiple vents disposed at spaced intervals and each formed through the surface layer; and
a cushioning layer disposed between the two surface layers and having
multiple cushioning layer fiber threads wound to form the cushioning layer, and each made of polyester; and
multiple openings disposed separately among the multiple cushioning layer fiber threads.

13. The medical device for support and fixation as claimed in claim 1, wherein the at least one supporting layer is a paper honeycomb.

14. The medical device for support and fixation as claimed in claim 1, wherein the medical device for support and fixation comprises a curing agent coated on the at least two three-dimensional fiber webs.

15. The medical device for support and fixation as claimed in claim 2, wherein the medical device for support and fixation comprises a curing agent coated on the two three-dimensional fiber webs.

16. The medical device for support and fixation as claimed in claim 1, wherein each one of the at least two three-dimensional fiber webs has four edges, and one of the four edges extends out of the at least one supporting layer and forms an extending portion overlapping with the extending portion of another one of the at least two three-dimensional fiber webs; the medical device for support and fixation comprises a label layer wrapped on the two overlapping extending portions.

17. The medical device for support and fixation as claimed in claim 1, wherein the medical device for support and fixation comprises multiple fixing elements, and each one of the multiple fixing elements is mounted to one of the at least two three-dimensional fiber webs; each one of the multiple fixing elements is connectable with a corresponding one of the multiple fixing elements by winding the medical device for support and fixation.

18. The medical device for support and fixation as claimed in claim 1, wherein the medical device for support and fixation comprises a supporting brace having
a bracing element vertically disposed on the supporting brace; and
a fastener mounted through the medical device for support and fixation and the bracing element and fixing the bracing element to the supporting brace.
